(19) United States
(12) Patent Application Publication
(10) Pub. No.: US 2008/0072896 A1
(43) Pub. Date: Mar. 27, 2008

(54) Multi-Level User Interface for a Breathing Assistance System

(75) Inventors: Bonny Carlene Setzer, Pleasanton, CA (US); Joseph Douglas Vandine, Newark, CA (US); William Brian Greger, San Francisco, CA (US)

Correspondence Address: Covidien;IP Counsel, Respiratory & Monitoring Solutions, 60 Middleton Avenue, North Haven, CT, 06473, US

(73) Assignee: Nellcor Puritan Bennett Incorporated

(21) Appl. No.: 11/535,964

(22) Filed: Sep. 27, 2006

(51) Int. Cl. A61M 16/00

(52) U.S. Cl. 128/200.24

(57) ABSTRACT

A ventilator system includes a ventilator configured for ventilating a patient based on a plurality of ventilation parameters, and a multi-level graphic user interface (GUI) coupled to the ventilator. The multi-level GUI may be configured to display a view menu allowing a user to select from multiple different views providing different levels of user access to the ventilation parameters. The multiple different views may include a first view and a second view. The first view may display values for a first set of ventilation parameters, and user access for adjusting the setting for at least one of the first set of the ventilation parameters. The second view may display values for a second set of the ventilation parameters, and access for adjusting the settings for at least one of the second set of the ventilation parameters. The second set of ventilation parameters may be larger than the first set of ventilation parameters.

### TECHNICAL FIELD

The present disclosure is related to breathing assistance systems, e.g., a multi-level user interface for a breathing assistance system.

### BACKGROUND

As ventilation systems and their various components (e.g., sensors and control systems) have become more sophisticated, and as more understanding is gained about the physiology of breathing and ventilatory therapy, the number of therapeutic alternatives and ventilator settings available to the caregiver has increased substantially. In addition, the interface between the ventilator and the ventilator user (e.g., caregiver or ventilation patient) has generally not been adaptable to the capabilities or sophistication of the user. For example, such interfaces often either limit the number of options or choices available for sophisticated users or present numerous options or choices that may confuse or overwhelm less sophisticated users.

In addition, clinical treatment of a ventilated patient often requires that the breathing characteristics of the patient be monitored to detect changes in the patient's breathing patterns. Many modern ventilators visually display various parameters regarding the patient's breathing patterns and/or the operation of the ventilator, and may allow the caregiver to adjust ventilator settings to fine tune the ventilation strategy being implemented. However, these systems are typically difficult for unsophisticated users to understand or use.

### SUMMARY

According to one embodiment of the present disclosure, a ventilator system may include a programmable ventilator and a multi-level graphic user interface (GUI) coupled to the ventilator. The ventilator may be configured for ventilating a patient based on a plurality of ventilation parameters. The multi-level GUI may be configured to display a view menu allowing a user to select from multiple different views providing different levels of user access to the ventilation parameters. The multiple different views may include a first view and a second view. The first view may display values for a first set of the ventilation parameters, each value comprising either a monitored value or a setting for a ventilation parameter. The first view may further provide user access for adjusting the setting for at least one of the first set of the ventilation parameters. The second view may display values for a second set of the ventilation parameters, each value comprising either a monitored value or a setting for a ventilation parameter. The second view may further provide access for adjusting the settings for at least one of the second set of the ventilation parameters. The second set of ventilation parameters may be larger than the first set of ventilation parameters.

According to another embodiment of the present disclosure, a multi-level graphic user interface (GUI) for use with a ventilator system is provided. The multi-level GUI may include a touch screen display configured to display a view menu allowing a user to select from multiple different views providing different levels of user access to a plurality of ventilation parameters. The multiple different views may include a first view and a second view. The first view may display values for a first set of the ventilation parameters, each value comprising either a monitored value or a setting for a ventilation parameter. The first view may further provide user access for adjusting the setting for at least one of the first set of the ventilation parameters. The second view may display values for a second set of the ventilation parameters, each value comprising either a monitored value or a setting for a ventilation parameter. The second view may further provide access for adjusting the settings for at least one of the second set of the ventilation parameters. The second set of ventilation parameters may be larger than the first set of ventilation parameters.

According to yet another embodiment of the present disclosure, logic may be provided for displaying a view menu allowing a user to select from multiple different views providing different levels of user access to a plurality of ventilation parameters. The multiple different views may include a first view and a second view. The first view may display values for a first set of the ventilation parameters, each value comprising either a monitored value or a setting for a ventilation parameter. The first view may further provide user access for adjusting the setting for at least one of the first set of the ventilation parameters. The second view may display values for a second set of the ventilation parameters, each value comprising either a monitored value or a setting for a ventilation parameter. The second view may further provide access for adjusting the settings for at least one of the second set of the ventilation parameters. The second set of ventilation parameters may be larger than the first set of ventilation parameters.

According to yet another embodiment of the present disclosure, a ventilator system includes ventilation means for ventilating a patient based on settings for a plurality of ventilation parameters, and display means for displaying a view menu allowing a user to select from multiple different views providing different levels of user access to the ventilation parameters. The multiple different views may include a first view and a second view. The first view may display values for a first set of the ventilation parameters, each value comprising either a monitored value or a setting for a ventilation parameter. The first view may further provide user access for adjusting the setting for at least one of the first set of the ventilation parameters. The second view may display values for a second set of the ventilation parameters, each value comprising either a monitored value or a setting for a ventilation parameter. The second view may further provide access for adjusting the settings for at least one of the second set of the ventilation parameters. The second set of ventilation parameters may be larger than the first set of ventilation parameters.

According to yet another embodiment of the present disclosure, a ventilator system includes a ventilator configured for ventilating a patient based on settings for a plurality of ventilation parameters, and a graphic user interface (GUI) including a touch screen display configured to display multiple different views. The multiple different views may provide different levels of user access for adjusting one or more of the ventilation parameters. The multiple different views may include at least one restricted access view. The GUI may be configured to manage user access to each restricted access view by requiring a user to enter particular input in order to access the restricted access view.

It should be understood that various changes, substitutions and alterations can be made herein without departing from the spirit and scope of the disclosure as illustrated by the following claims.

### BRIEF DESCRIPTION OF THE DRAWINGS

Some embodiments of the disclosure may be understood by referring, in part, to the following description and the accompanying drawings, in which like reference numbers refer to the same or like parts and wherein:

FIG. 1 illustrates a ventilation system for providing ventilatory therapy to a patient, according to one embodiment of the disclosure;

FIG. 2 is a block diagram illustrating various components of a multi-level GUI and a ventilator of the ventilation system of FIG. 1, according to an example embodiment;

FIG. 3 illustrates an example configuration of a multi-level GUI module for use with a ventilator, according to an example embodiment;

FIG. 4 illustrates an example layout of a touch screen GUI display, according to certain embodiments of the disclosure;

FIG. 5 illustrates an example of a simple view generated and displayed by a multi-level GUI, according to an embodiment of the disclosure;

FIG. 6 illustrates an example of a main view generated and displayed by a multi-level GUI, according to an embodiment of the disclosure;

FIG. 7 illustrates an example of an alarm condition displayed in the main view of FIG. 6, according to an embodiment of the disclosure;

FIG. 8 illustrates an example of an advanced-gauge view generated and displayed by a multi-level GUI, according to an embodiment of the disclosure;

FIG. 9 illustrates an example of an alarm condition displayed in the advanced-gauge view of FIG. 8, according to an embodiment of the disclosure;

FIG. 10 illustrates an example of an advanced-waveform view generated and displayed by a multi-level GUI, according to an embodiment of the disclosure;

FIG. 11 illustrates an example of an alarm condition displayed in the advanced-waveform view of FIG. 10, according to an embodiment of the disclosure;

FIG. 12 illustrates an example GUI view having a menu region displaying various menu items that may be selected by a user, according to an embodiment of the disclosure;

FIG. 13 illustrates an example view in which a view menu button has been selected by a user, according to an embodiment of the disclosure;

FIG. 14 illustrates an example view in which a main menu button has been selected by a user, according to an embodiment of the disclosure;

FIGS. 15-21 illustrate menus and functions associated with a preset menu button, according to an embodiment of the disclosure; and

FIG. 22 illustrates an example view in which a breath mode menu button has been selected by a user, according to an embodiment of the disclosure.

### DETAILED DESCRIPTION

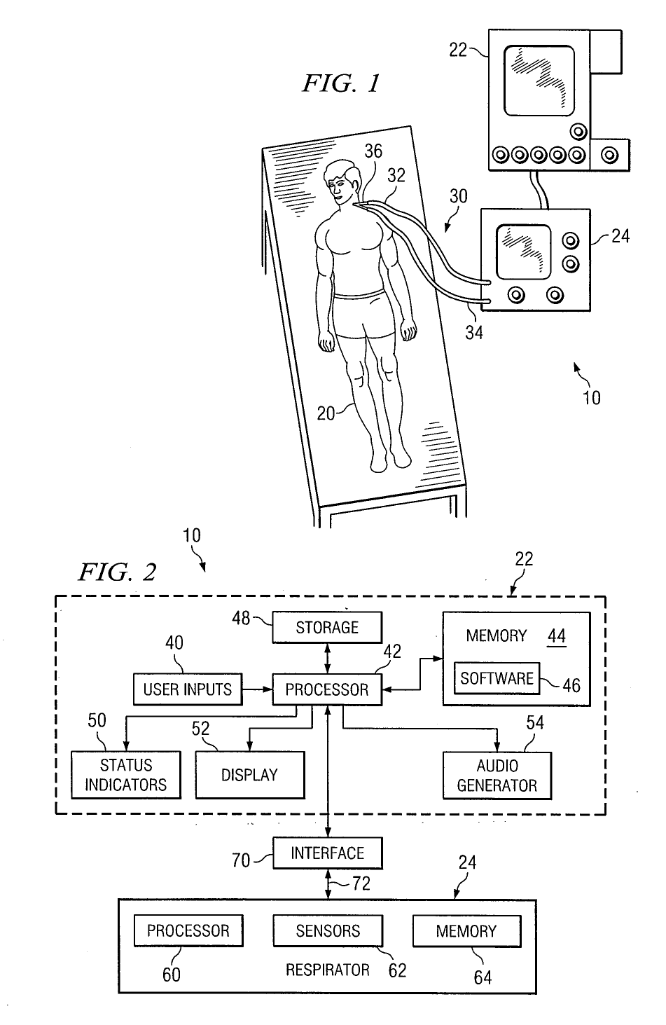

Selected embodiments of the disclosure may be understood by reference, in part, to FIGS. 1-22. The present disclosure relates generally to user interfaces for breathing assistance systems. In some embodiments, a multi-level graphic user interface (GUI) for a breathing assistance system (e.g., a ventilator, CPAP device, or BiPAP device) is provided that may provide different views that may be appropriate for and/or understandable by users of different levels of sophistication regarding ventilatory therapy. The different views displayed by the GUI may provide different levels of access to view, select, and/or adjust monitored values or settings for various ventilation parameters. Because users having different levels of sophistication typically have different needs for information displays and access to controls, e.g., ranging from relatively basic displays and/or controls for relatively unsophisticated users (e.g., non-healthcare professional caregivers) to relatively complex displays and/or controls for relatively advanced users (e.g., respiratory therapists), the multiple levels of access provided by the GUI may provide different views of ventilation information to support the different wants or needs of different types of users, and/or to ensure immediacy of the information. In some embodiments, the display may include a touch screen display allowing a user to view, select, and/or adjust settings for various parameters by touching the screen.

Each of the different views displayed by GUI may display values for different sets of ventilation parameters. Each displayed value may be either a monitored value for the parameter (e.g., a pressure detected by a sensor) or a setting for the parameter (e.g., a setting manually selected by a user or automatically implemented by ventilation software). A view may display monitored values for one or more first ventilation parameters and settings for one or more second ventilation parameters, where one or more of the first ventilation parameters and second ventilation parameters may be the same parameters.

In some embodiments, the GUI may display multiple views providing different levels of access to ventilation parameters. For example, some views may display monitored values only; other views may display monitored values and settings for a set of ventilation parameters, but not provide user access to adjust such settings; other views may display monitored values and settings for a set of ventilation parameters, and provide user access to adjust one or more of such settings.

In some embodiments, views may be classified by level of access to ventilation parameters, such as:

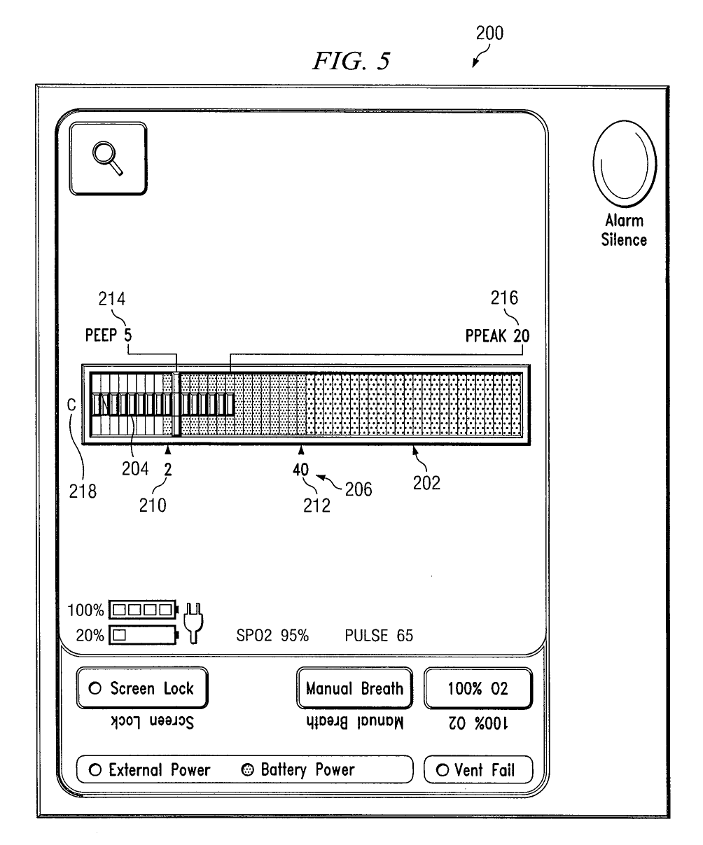

(1) A first level of user access (Level 1) generally provides the user access to view values (monitored values and/or settings) for one or more ventilation parameters, but may not provide access for adjusting settings. Thus, a Level 1 access view may display monitored values (e.g., monitored values for pressure and/or flow) and/or settings for one or more ventilation parameters, but may provide no access for adjusting such settings. Views classified as Level 1 access may be used in environments in which minimizing the displayed data is desired or necessary. In the embodiments discussed herein, the Simple view shown in FIG. 5 may be classified as a Level 1 access view.

(2) A second level of user access (Level 2) generally provides the user access to view values (monitored data and/or settings) for a first set of ventilation parameters, and may provide access for adjusting settings for one or more of the first set of ventilation parameters. The first set of ventilation parameters may include frequently monitored or adjusted ventilation parameters (e.g., respiratory rate, pressure, minute volume, and/or tidal volume), but may not include more complicated or advanced parameters. Thus, a Level 2 access view may display monitored values (e.g., monitored values for pressure and/or flow) as well as settings for a first set of ventilation parameters, and may provide access for adjusting one or more of such settings.

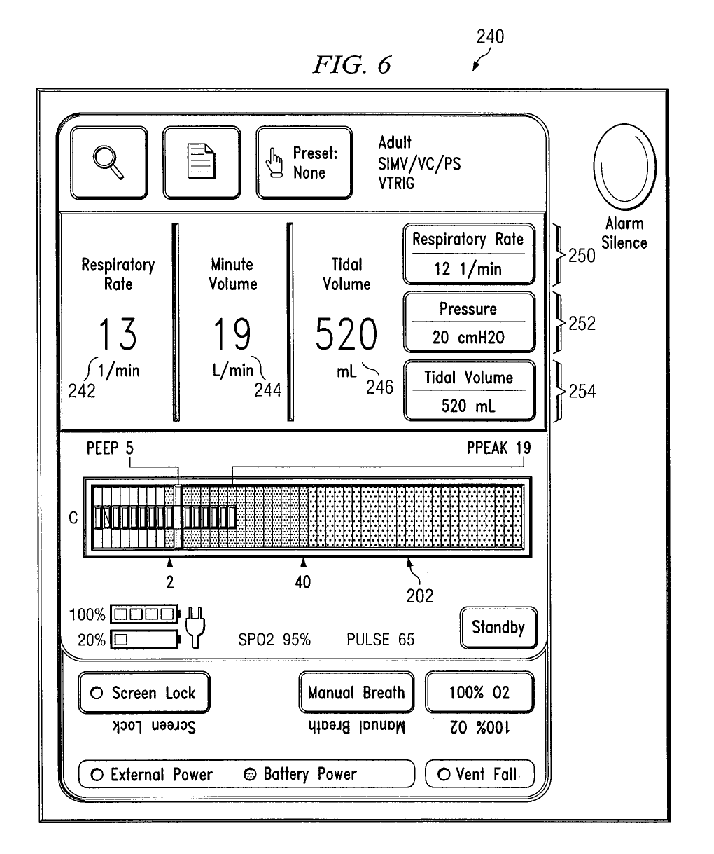

In some embodiments, Level 2 access views may provide one-step control of settings for one or more ventilation parameters (e.g., respiratory rate, pressure, and/or tidal volume). According to such one-step control, a user may touch a one-touch icon (e.g., button) corresponding to the parameter to be adjusted, which may bring up a pop-up window for setting or adjusting the parameter as desired, and then return the user to the previous view once the selection or adjustment has been completed. In this manner, the user may avoid navigating through more complicated menus and/or adjusting more complicated parameters. In the embodiments discussed herein, the Main view shown in FIG. 6 may be classified as a Level 2 access view.

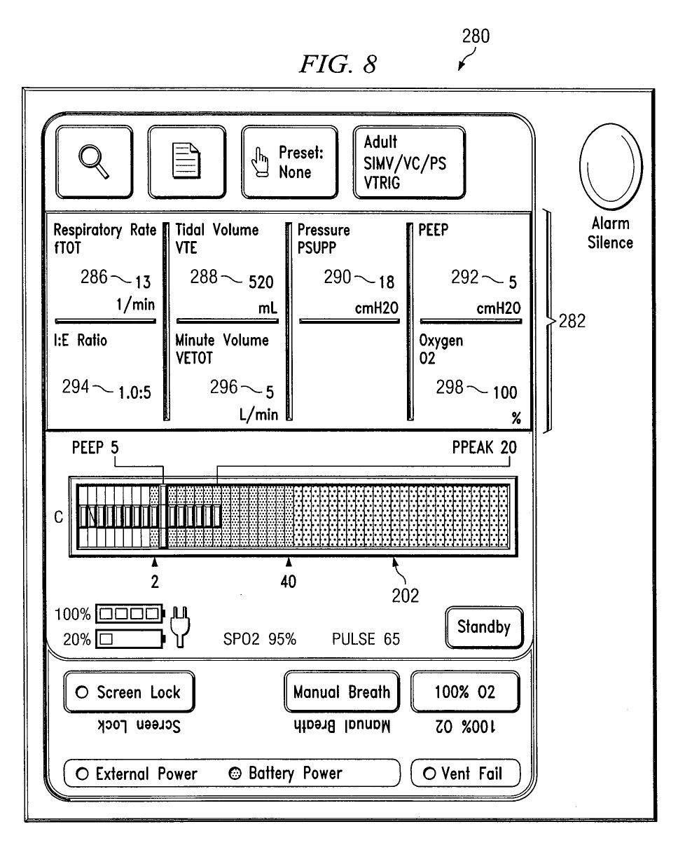

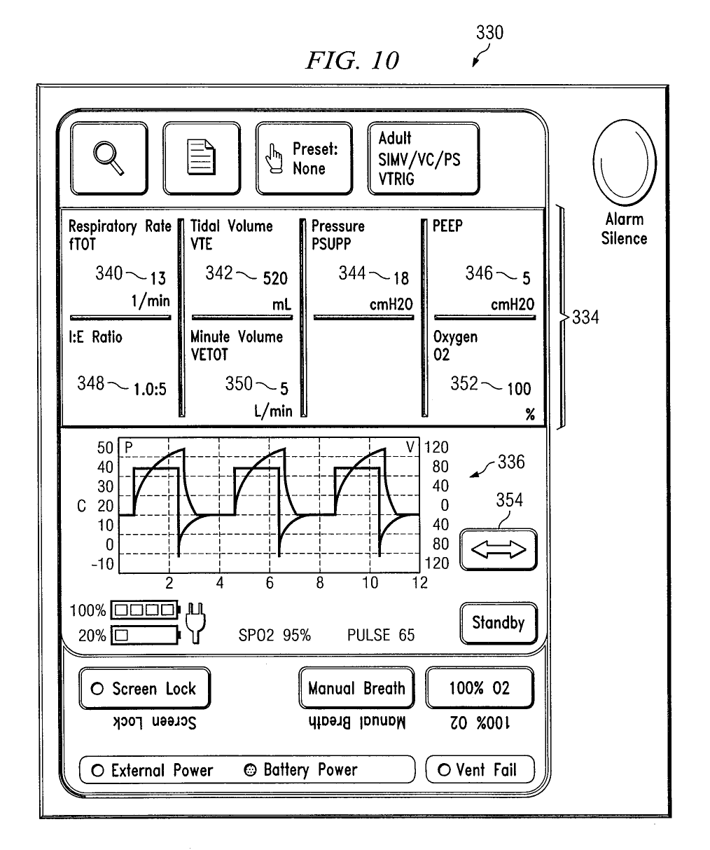

(3) A third level of user access (Level 3) generally provides the user access to view values (monitored data and/or settings) for a second set of ventilation parameters, and may provide access for adjusting settings for one or more of the second set of ventilation parameters. The second set of ventilation parameters may be generally more comprehensive or advanced than the first set of ventilation parameters accessible in a Level 2 access view. For example, the second set of ventilation parameters may include one or more relatively complex or advanced parameters, e.g., parameters that would typically be viewed or adjusted by a medical professional (e.g., a respiratory therapist). Thus, a Level 3 access view may display monitored values (e.g., monitored values for pressure and/or flow) as well as settings for a second set of ventilation parameters (which may be more comprehensive or advanced than those accessible in a Level 2 access view), and may provide access for adjusting one or more of such settings. Level 3 access views may be used, e.g., by sophisticated users who are comfortable with advanced or complex ventilation parameters. In the embodiments discussed herein, the Advanced-Gauge and Advanced-Waveform views shown in FIGS. 8 and 10, respectively, may be classified as a Level 3 access views.

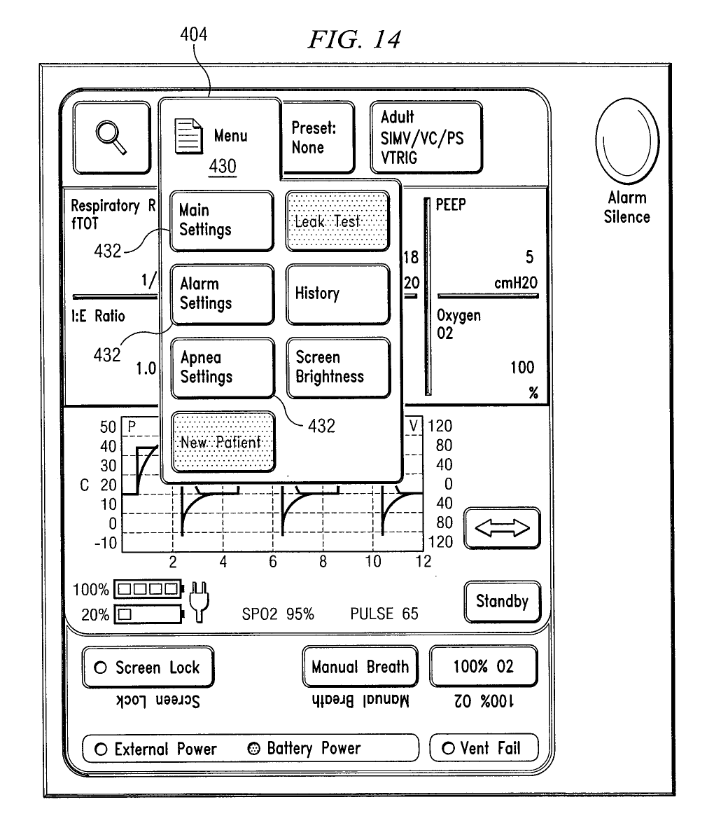

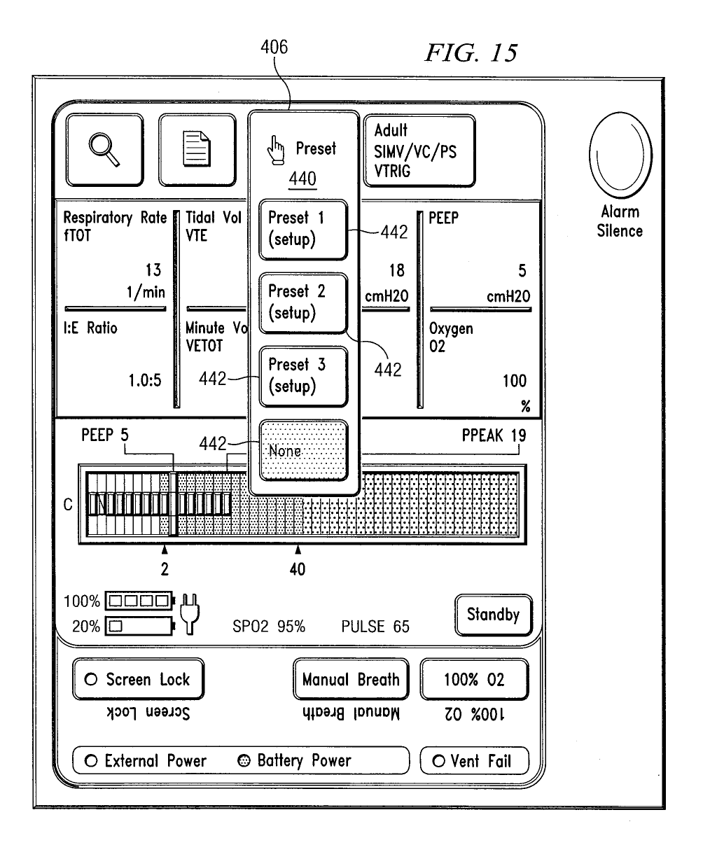

One or more views displayed by the GUI may include a menu icon (e.g., a menu button) that may be selected to provide user access to a set of settings and/or other data. For example, in an embodiment discussed below regarding FIG. 14, a menu of settings and/or other data may include one or more of: main settings, alarm settings, apnea settings, a leak test, new patient set-up, screen brightness adjustment, and history/alarm logs.

In some embodiments, the menu of settings and/or other data that may be accessed via the menu icon may depend on the access level of the particular view. For example, selecting the menu icon in a Level 1 access view may provide the user access to a first menu of settings and/or other data, selecting the menu icon in a Level 2 access view may provide the user access to a second menu of settings and/or other data larger than the first menu of settings and/or other data, and selecting the menu icon in a Level 3 access view may provide the user access to a third menu of settings and/or other data larger than the second menu of settings and/or other data. As discussed below regarding FIG. 14, in some embodiments, icons corresponding to particular settings and/or other data that are not accessible in a particular view may be grayed out or hidden from the menu displayed when the menu icon is selected in that view.

In some embodiments, any user may access any view displayed by the GUI, e.g., by selecting any view from a view menu. In other embodiments, the GUI may manage user access to particular views, thereby managing user access to access particular values, modify particular settings, or access other data. For example, the GUI may restrict user access to particular views using any suitable restriction technique, e.g., using passwords or access keys, or requiring particular buttons or icons to be pressed simultaneously or in sequence.

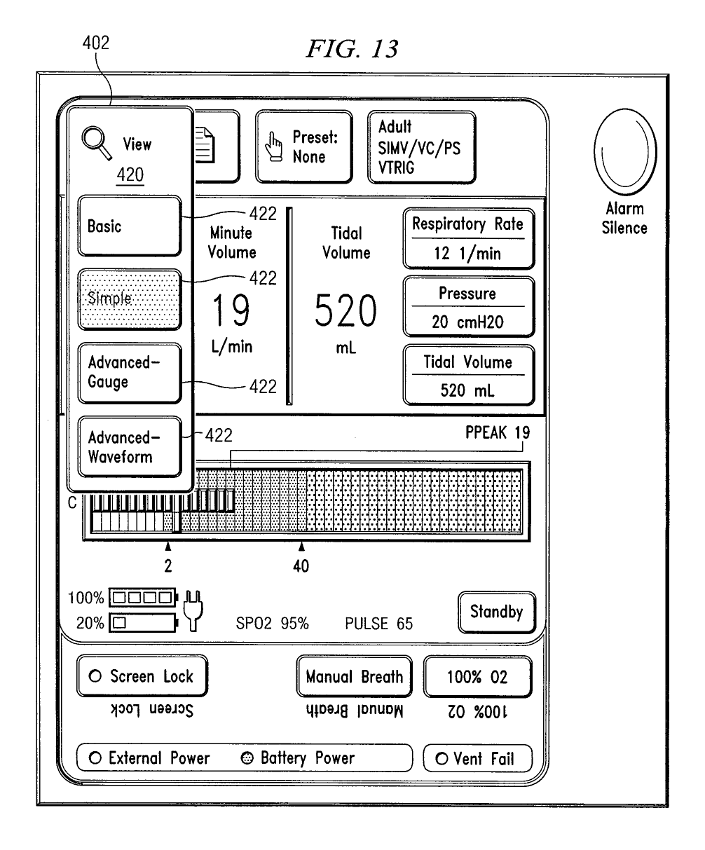

In some embodiments, one or more views may have restricted access, while one or more other views may have open or unrestricted access. For example, as discussed below with reference to FIG. 13, when a user selects an unrestricted access view from a view menu, the unrestricted access view may be displayed. However, when a user selects a restricted access view from the view menu, the GUI may require the user to bypass the restriction in order to display the restricted access view. For example, the GUI may require the user to enter a password or access key, or may require the user to pressed particular buttons or icons simultaneously or in sequence. In this manner, the GUI may restrict access to particular settings and/or data to particular users.

In some embodiments, the ventilator or GUI may include a housing that includes one or more of the following: a control device for silencing an alarm for a predetermined period of time or for resetting an alarm; a control device for deactivating user interaction with the touch screen display; a control device for causing the ventilator to initiate a breath according to current breath settings of the programmable ventilator controller; a control device for initiating delivery of 100% oxygen to the patient for a predetermined period of time; an indicator of a source of power of the ventilator; and/or an indicator for indicating a malfunction of the ventilator or related hardware or software.

As discussed above, the GUI may display a plurality of graphic menus. Such menus may include one or more of the following: a views menu enabling the user to select from multiple different views; a main menu allowing the user to access various settings (e.g., ventilation parameter settings and/or other settings) and/or other data; a presets menu providing the user access to select a particular preset ventilation program or therapy to implement and/or to setup or edit one or more preset ventilation programs or therapies; and a breath settings menu for allowing the user to adjust a current breath mode, a breath type, and/or one or more breath trigger options.

As used herein, the term “ventilator” may refer to any device, apparatus, or system for delivering breathing gas to a patient, e.g., a ventilator (e.g., a critical care ventilator or a home use ventilator), a CPAP device, or a BiPAP device. The term “patient” may refer to any person or animal that is receiving breathing support from a ventilator, regardless of the medical status, official patient status, physical location, or any other characteristic of the person. Thus, for example, patients may include persons under official medical care (e.g., hospital patients), persons not under official medical care, persons receiving care at a medical care facility, persons receiving home care, etc.

FIG. 1 illustrates a ventilation system 10 for providing ventilatory therapy to a patient 20, according to an embodiment of the disclosure. Ventilation system 10 may include a multi-level graphic user interface (GUI) module 22 connected to and operable to receive input for controlling a breath delivery apparatus (or ventilator) 24, and a patient circuit 30 for connecting ventilator 24 to patient 20. Patient circuit 30 may include an inspiration line 32, an expiration line 34, and/or a patient connection tube 36, which may be connected by a patient connector, as known in the art.

FIG. 2 is a block diagram illustrating various components of multi-level GUI module 22 and ventilator 24 of ventilation system 10, according to an example embodiment. Multi-level GUI module 22 may include one or more user inputs 40, a processor 42, memory 44, status indicators 50, a display 52, and/or an audio generator 54. User inputs 40 may include any suitable interface(s) allowing a user to input data to GUI module 22, such as one or more user controls 80 and a touch screen display 52 (discussed in greater detail below). Processor 42 may include a microprocessor, a microcontroller, a digital signal processor (DSP), an application specific integrated controller (ASIC), electrically-programmable read-only memory (EPROM), or a field-programmable gate array (FPGA), or any other suitable processor(s), and may be generally operable to provide processing related to the operation of GUI module 22. Memory 44 may comprise any one or more devices suitable for storing electronic data, e.g., RAM, DRAM, ROM, one or more hard disks, and/or any other memory or storage device. Memory 44 may be used to store, for example, current settings, system status, patient data, and/or GUI software 46. GUI software 46 may include any code or logic that may be executed by processor 42 and/or other processor(s) in order to provide any of the functionality of multi-level GUI module 22 discussed herein.

Processor 42 may also be connected to a storage device 48, e.g., battery protected memory, a hard drive, a floppy drive, a magnetic tape drive, and/or other storage media for storing, e.g., patient data and/or associated ventilator operating parameters. Processor 42 may accept input received from user inputs 40 to control ventilator 24. Status indicators 50 may be generally configured to indicate the status of various parameters related to ventilation system 10 and/or patient 20. Display 52 may be generally configured to display patient data and/or ventilator settings. As discussed herein, display 52 may be an interactive display (e.g., a touch screen) that allows a user to view, select, set, adjust, and/or otherwise manage one or more parameters related to ventilation system 10 and/or patient 20. Audio generator 54 may be generally configured to provide audible indications of the status of ventilation system 10 and/or patient 20.

Ventilator 24 may include a processor or controller 60, one or more sensors 62, and/or memory 64. Processor or controller 60 may include a microprocessor, a microcontroller, a digital signal processor (DSP), an application specific integrated controller (ASIC), electrically-programmable read-only memory (EPROM), or a field-programmable gate array (FPGA), or any other suitable processor(s) or hardware, and may be generally operable to control the operation of ventilator 24, including various breath delivery functions.

In some embodiments, multi-level GUI module 22 may be partially or fully integrated (e.g., physically, electronically, and/or via wireless communications) with ventilator 24. For example, processors 42 and 60 may be the same processor or may be integrated, and memories 44 and 65 may be the same memories or may be integrated. In other embodiments, multi-level GUI module 22 may be partially or completely distinct (e.g., detachable, removable, or permanently separate) from ventilator 24. For example, GUI module 22 may include a housing that sits on top of ventilator 24. In such embodiments, GUI module 22 may be coupled to ventilator 24 by an interface 70. Interface 70 may communicate various data between ventilator 24 and GUI module 22. For example, interface 70 may communicate control signals from processor 42 of GUI module 22 to processor 60 of the ventilator 24, and/or may receive signals from sensors 62 associated with ventilator 24 indicative of the status of patient 20 and/or ventilator 24. Interface 70 may include, for example, an Ethernet connection, an RS-232 serial interface, or a wireless interface. A cable 72 having an appropriate number of conductors may be used to connect ventilator 24 to an appropriate connector of interface 70. In other embodiments, GUI module 22 may connect directly to ventilator 24, e.g., via a suitable cable.

In some embodiments, memory 44 and/or memory 64 may be, for example, non-volatile random access memory (NVRAM) for storing important, persistent variables and/or configuration settings, such as current breath mode setup. Typically, during normal operation of ventilation system 10, such NVRAM may function similar to a typical random access memory. If, however, a low-voltage condition is detected, such as may occur during a brown-out or at the beginning of a power failure, for example, the NVRAM may automatically store its data in non-volatile storage.

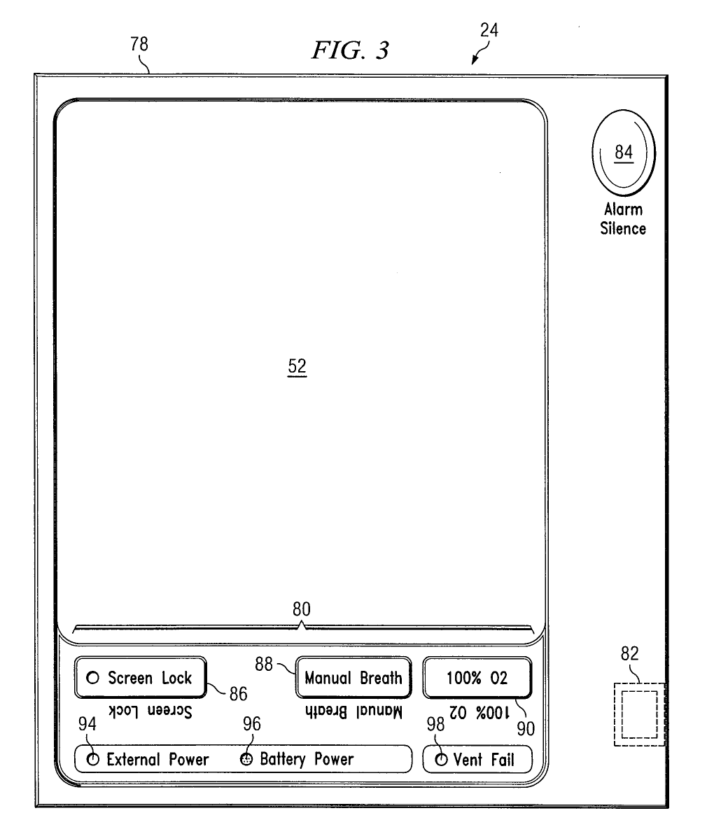

FIG. 3 illustrates an example configuration of a multi-level GUI module 22 for use with a ventilator 24, according to an example embodiment. In this embodiment, GUI module 22 may include a housing 78 and various user inputs 40, including one or more user controls 80 and a multi-level display 52. Multi-level display 52 may be communicatively connected to processor 42 for displaying a number of ventilation parameters and/or menus enabling the user to select from multiple views of various ventilation parameters, as discussed below in greater detail. In some embodiments, display 52 may comprise a touch screen display, which may operate as a user input 40 allowing the user to make various selections, e.g., selecting a particular view, selecting particular ventilation parameters, and/or setting or adjusting particular ventilation parameters.

As used herein, a “ventilation parameter” may include any parameter regarding the operation of ventilation system 10, ventilation assistance provided to patient 20 (e.g., parameters regarding the delivery of gas to patient 20), physical or other characteristics of patient 12, the environment, and/or any other parameters regarding ventilation system 10 and/or patient 12.

One or more user controls 80 may be coupled to or integrated with housing 78. User controls may include, e.g., a power button 82, an alarm silence/reset button 84, a screen lock button 86, a manual breath button 88, and/or a 100% O₂ button 90. One or more of the user controls 80 may be provided for (a) critical ventilator functions that should be continuously available and/or (b) functions that are not to be controlled via touch screen display 52, e.g., a screen lock function.

Power button 82 may be on a side panel of housing 78, rather than on the front of housing 78, and/or may be recessed to help prevent accidental actuation. In one embodiment, to turn on ventilator 24, the user presses and holds power button 82 for an extended period of time, e.g., three seconds. Similarly, to turn off ventilator 24, the user presses and holds power button 82 for an extended period of time, e.g., three seconds. Before ventilator 24 shuts down, a confirmation screen may be displayed asking the user to confirm that ventilator 24 should be turned off.

Alarm silence/reset button 84 may be located in a corner (e.g., an upper right hand corner) of housing 78. The alarm silence/reset button may be, for example, a relatively large, translucent button with an LED (e.g., a red LED) that flashes when an alarm condition is present. Pressing alarm silence/reset button 84 may silence an alarm for a predetermined period of time, e.g., for two minutes. However, alarm silence/reset button 84 may continue to flash or remain on until the alarm is reset, e.g., by again pressing alarm silence/reset button 84. Alarm silence/reset button 84 may also be used to silence an alarm preemptively, for a predetermined period of time, e.g., for two minutes.

Screen lock button 86 may act as a safeguard against accidental actuation of buttons or other inputs on display 52. In some embodiments, display 52 can be locked manually using button 86 and/or may be set for automatic locking after a period of inactivity, e.g., two minutes. To activate the screen lock, a user may press and hold screen lock button 86 for a predetermined amount of time, e.g., three seconds. In one embodiment, an LED inside screen lock button 86 may then turn on to indicate that the screen is locked. To deactivate the screen lock, the user can again press and hold screen lock button 86 for a predetermined amount of time, e.g., three seconds. When the screen is unlocked, the screen lock button LED may turn off. In addition, in some embodiments, if display 52 is in the locked state and an on-screen button or keypad button is touched, a warning message may be displayed on the display 52 that informs the user that the screen lock is activated.

Manual breath button 88 may be pressed to cause ventilator 24 to deliver one breath (or more than one breath) according to current mandatory breath settings for the patient. The 100% O₂ button 90 may be pressed to cause ventilator 24 to deliver 100% oxygen to the patient for a predetermined period of time, e.g., if an oxygen concentrator feature is enabled for the ventilator.

One or more LEDs on the ventilator's housing 78 may work in conjunction with one or more audible indicators, hardware buttons, and/or on-screen information on display 52 to provide redundant feedback regarding the state of ventilator 24 and/or the power source(s). One or more power source LEDs may indicate the source from which ventilator 24 is currently drawing power. For example, if ventilator 24 is plugged into an AC power source (e.g., a wall outlet or a cigarette lighter), an external power LED 94 may light up. If ventilator 24 is running on batteries, a battery power LED 96 may light up.

A vent fail LED 98 may light up red when ventilator 24 is experiencing a major mechanical malfunction or if display 52 or various other system hardware and/or software components fail. Vent fail LED 98 may signal a catastrophic failure of the ventilator such that it cannot function at all. In some embodiments, vent fail LED 98 does not light up during routine alarms, such as high pressure or a disconnect, for example.

As discussed above, multi-level GUI module 22 may generate and display multiple different views on a touch screen display 52. The different views may have different levels of complexity and/or provide different levels of access to ventilation parameters. For example, different views may display values for different sets of ventilation parameters and/or allow users to adjust settings for different sets of ventilation parameters. The different views may be appropriate for, or correspond to, users having various levels of sophistication regarding ventilatory care, such as, for example, doctors, nurses, respiratory therapists, home care providers, medical equipment representatives, and/or ventilation patients (i.e., persons receiving the ventilatory care). The different views may allow the user to pick the view that includes particular information that the user wants or needs to view or monitor, e.g., based on the sophistication of the user, the particular patient being treated, the type of care being provided, and/or the personal preferences of the user.

In an example embodiment, multi-level GUI module 22 may generate and display four different views on touch screen display 52, including:

1. A Simple View (Level 1 access) (see, e.g., FIG. 5)—this view may display monitored ventilation data (e.g., an airway graphic indicating monitored pressure and/or flow data) and/or one or more ventilation parameter settings, but may suppress a significant amount of monitored patient data (e.g., data typically understood or used by relatively sophisticated users) and may provide no access for adjusting ventilation parameter settings.

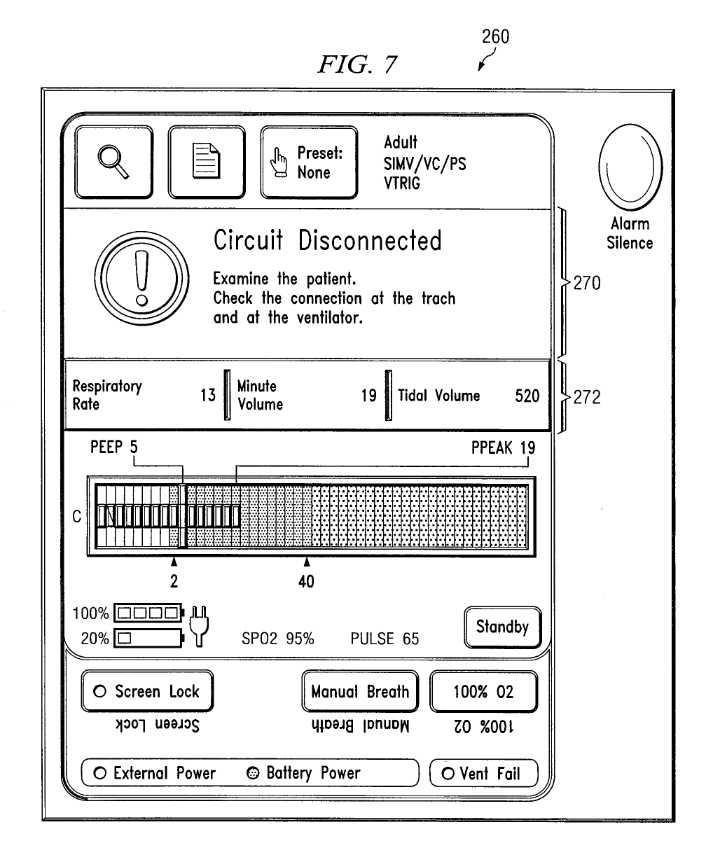

2. A Main View (Level 2 access) (see, e.g., FIGS. 6-7)—this view may display monitored ventilation data (e.g., an airway graphic indicating monitored pressure and/or flow data) and settings for a first set of ventilation parameters, and may provide access for adjusting one or more of such settings. In some embodiments, the first set of ventilation parameters may include parameters that are frequently monitored in a home care environment. The Main View may also include a number of one-touch icons allowing a user to easily select and/or adjust the settings for particular ventilation parameters.

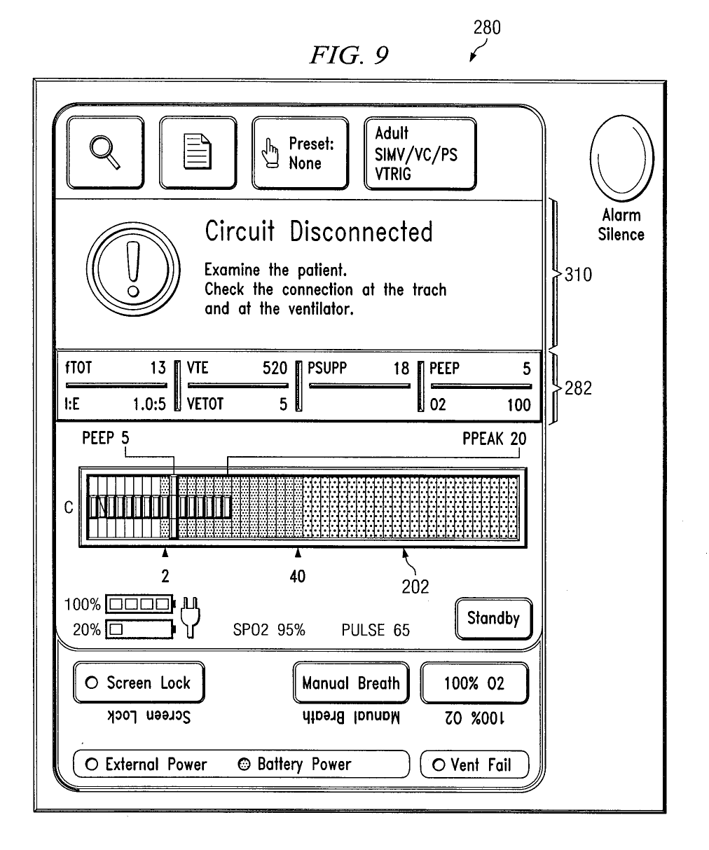

3. An Advanced-Gauge View (Level 3 access) (see, e.g., FIGS. 8-9)—this view may display (a) a gauge graphic and (b) settings for a second set of ventilation parameters, and may provide access for adjusting one or more of such settings. The gauge graphic may include a gauge having an indicator that dynamically advances and retreats to indicate at least one of monitored pressure data and monitored flow data. The second set of ventilation parameters may be more comprehensive and may include more advanced parameters than the first set of ventilation parameters. In some embodiments, the second set of ventilation parameters may include one or more relatively complex or advanced parameters, e.g., parameters that would typically be viewed or adjusted by a medical professional (e.g., a respiratory therapist).

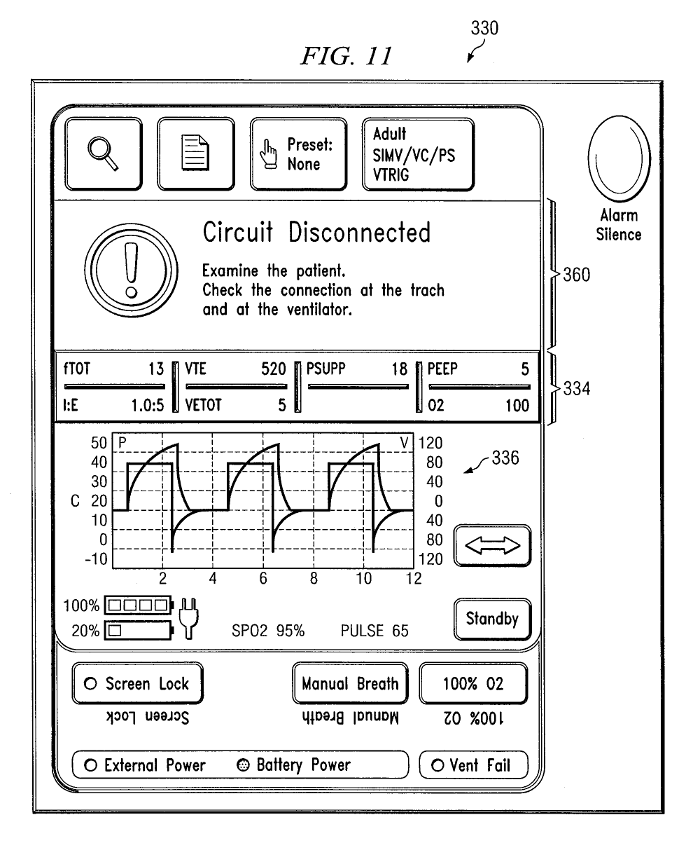

4. An Advanced-Waveform View (Level 3 access) (see, e.g., FIGS. 10-11)—this view may be similar to the Advanced-Gauge View, but may include a waveform graphic instead of a gauge graphic. The waveform graphic may include a graphical waveform indicating monitored pressure data and/or monitored flow data.

These example views are discussed in greater detail below. In other embodiments, GUI module 22 may generate and display other numbers (e.g., more or less than four) and/or different types of views.

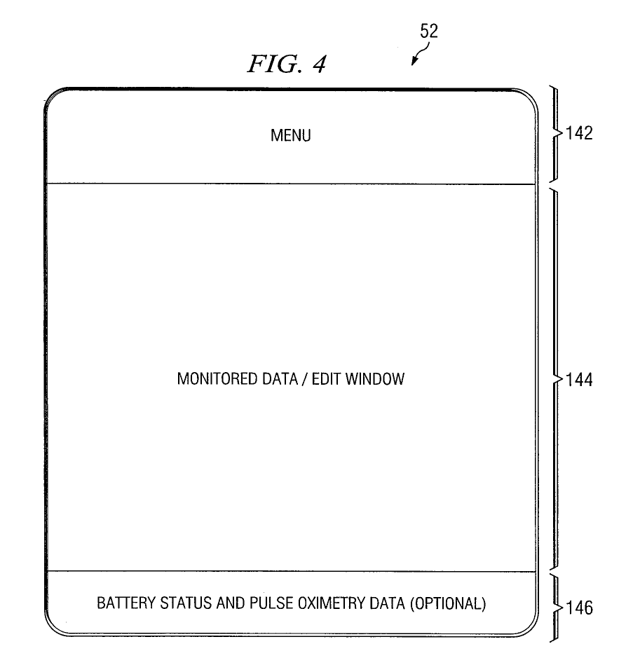

FIG. 4 illustrates an example layout of views displayed by touch screen display 52 of GUI module 22, according to certain embodiments of the disclosure. Each of the different views may have the same general layout, which may include a menu region 142, a monitored data and/or parameter control region 144, and in some embodiments, a battery status and/or pulse oximeter data region 146.

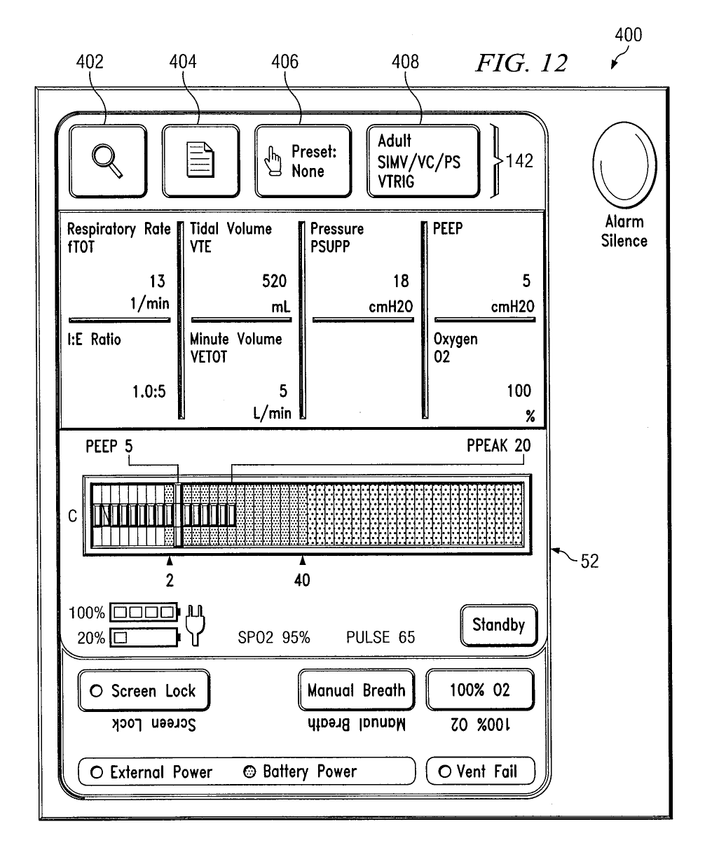

Menu region 142 may provide various menu items that may be selected by a user, for example, to access the different views; access various settings; set up a new patient for ventilatory care; view history and/or alarm logs; setup, edit and/or view multiple preset breath delivery therapies; and/or adjust the current breath mode, breath type, and/or breath trigger options. In some embodiments, a user may touch display 52 to make selections from menu region 142. Various aspects of menu region 142 may be better understood in view of FIGS. 12-22, discussed below.

Monitored data and/or parameter control region 144 may generally display values (e.g., monitored values and/or settings) for one or more ventilation parameters, e.g., the patient's airway pressure, a flow volume, the patient's respiratory rate, an I:E ratio, PEEP, and/or an O₂ percentage. In some embodiments, a user may touch display 52 to select and/or adjust settings for particular ventilation parameters displayed in region 144. The monitored values and/or settings may be presented in varying degrees of complexity based on the user or caregiver's needs or preferences. Depending on the embodiment and/or particular display, such data may be fully graphical, mostly graphical and partly digital (represented using numerical digits), mostly digital and partly graphical, or fully graphical.

Battery status and/or pulse oximeter data region 146 may display the status of one or more ventilator batteries (e.g., percent charged) and/or various data received from a pulse oximeter connected to the patient. The battery charge indicators may provide the current status of the battery charge levels. The pulse oximetry data may indicate the currently monitored oxygen saturation and/or pulse rate data. In some embodiments, a user may touch display 52 to select and/or adjust particular parameters displayed in region 146. In some embodiments, region 146 may be optional.

FIG. 5 illustrates an example of a simple view 200 generated and displayed on display 52 by multi-level GUI module 22, according to an embodiment of the disclosure. Simple view 200 may be appropriate for, or correspond to, a user (e.g., a caregiver) that wants to suppress a significant portion of the monitored patient data (e.g., data typically understood and/or used by relatively sophisticated users) but still have a visual indication that ventilation is occurring. View 200 may include a patient airway pressure graphic 202 representing the patient airway pressure, e.g., in real time. In this example, graphic 202 includes a graphic bar 204 that advances and retreats according to the current patient airway pressure. In some embodiments, graphic 202 may be seen from a distance and may thus provide the user an assurance at a glance that ventilator 24 is delivering breaths.

Patient airway pressure graphic 202 may include a scale 206 indicating various ventilation parameter settings, including a minimum pressure threshold setting 210, a maximum pressure threshold setting 212, and a Positive End Expiratory Pressure (PEEP) setting 214. A Peak Pressure (P-Peak) value 216 may also be captured and displayed on a breath-to-breath basis. The type of breath that is being delivered may be indicated at the front of graphic 202 with an “C” for a machine controlled breath, “S” for a Spontaneous breath, or “A” for an Assisted breath, as indicated at 218. In some embodiments, simple view 200 does not provide the user access to adjust the settings for the displayed ventilation parameters.

FIG. 6 illustrates an example of a main view 240 generated and displayed on display 52 by multi-level GUI module 22, according to an embodiment of the disclosure. Main view 240 may be appropriate for, or correspond to, various types of users, e.g., a home care-giver or a durable medical equipment representative. In some embodiments, view 240 may provide access to ventilation parameters that are frequently monitored in a particular ventilation environment (e.g., a home care environment), such as access to monitored values and/or settings for respiratory rate, minute volume, tidal volume, and/or pressure, for example. For example, as shown in FIG. 6, real-time monitored values for respiratory rate, minute volume, and tidal volume are indicated respectively at 242, 244, and 246, and settings for respiratory rate, pressure, and tidal volume are indicated respectively at 250, 252, and 254. The monitored values to be displayed in view 240 may be predefined or selected by the user. In addition, in some embodiments, the data may be presented to the user in full English terminology (and/or translated into one or more foreign languages), rather than using ventilation symbols.

Main view 240 may provide one-step control of various ventilation parameters (e.g., respiratory rate, pressure, and tidal volume). For example, as shown in FIG. 6, main view 240 may include one-touch icons 250, 252, and 254 for adjusting the settings for respiratory rate, pressure, and tidal volume. When a particular one-touch icons 250, 252, or 254 is selected by a user, a pop-up window may be displayed, which may provide buttons or other icons for setting or adjusting the particular parameter as desired. Once the user has completed the adjustment, the pop-up window may close. In this manner, main view 240 may give the user direct access, or shortcuts, to particular settings that may be frequently accessed, thus allowing the to avoid navigating through more complicated menus and/or adjusting more complicated parameters. Thus, for example, a home caregiver may avoid having to navigate to a full settings screen that may include parameters and symbols with which they may be unfamiliar. In addition, view 240 may include a patient airway pressure graphic 202, as discussed above with respect to FIG. 5.

FIG. 7 illustrates an example of an alarm condition displayed in main view 260, according to an embodiment of the disclosure. During an alarm condition, an alarm message window 270 may be displayed in the same location as the digital monitored data (i.e., region 144 discussed above regarding FIG. 4). In some embodiments, while alarm message window 270 is active, the digital monitored data (indicated at 272) may still be displayed but resized to a smaller window or area.

FIG. 8 illustrates an example of an advanced-gauge view 280 generated and displayed on display 52 by multi-level GUI module 22, according to an embodiment of the disclosure. Advanced-gauge view 280 may be appropriate for, or correspond to, relatively sophisticated users, e.g., respiratory therapists. In some embodiments, view 280 may provide access to a more comprehensive and/or advanced set of ventilation parameters than main view 240 or simple view 200. View 280 may display a patient airway pressure graphic 202 and values (e.g., monitored values and/or settings) for a relatively advanced set of ventilation parameters, and may provide access for adjusting settings for one or more of such ventilation parameters. The second set of ventilation parameters may be more comprehensive and may include more advanced parameters than the first set of ventilation parameters. In some embodiments, the second set of ventilation parameters may include one or more relatively complex or advanced parameters, e.g., parameters that would typically be viewed or adjusted by a medical professional (e.g., a respiratory therapist).

In the embodiment shown in FIG. 8, view 280 may present the user with monitored patient data 282, as well as patient airway pressure graphic 202. In this example embodiment, monitored patient data 282 includes monitored values for the patient's respiratory rate 286, a tidal volume 288, a pressure support value 290, PEEP 292, an I:E ratio 294, a minute volume 296, and an O₂ percentage 298. One or more of the monitored patient data 282 to be displayed in view 280 may be predefined or selected by the user. In addition, one or more of the monitored values may correspond to settings for the particular parameters. For example, for some ventilation parameters, the breath delivery apparatus 24 will implement the settings for such ventilation parameters such that the monitored values are the same as the settings.

FIG. 9 illustrates an example of an alarm condition displayed in advanced-gauge view 280, according to an embodiment of the disclosure. During an alarm condition, an alarm message window 310 may be displayed in the same location as the digital monitored data (i.e., region 144 discussed above regarding FIG. 4). In some embodiments, while alarm message window 310 is active, the monitored patient data 282 may still be displayed, but resized to a smaller window or area.

FIG. 10 illustrates an example of an advanced-waveform view 330 generated and displayed on display 52 by multi-level GUI module 22, according to an embodiment of the disclosure. Advanced-waveform view 330 may be appropriate for, or correspond to, relatively sophisticated users, e.g., respiratory therapists. Advanced-waveform view 330 may be similar to the Advanced-Gauge View, but may include a pressure and flow waveform graphic 336 instead of patient airway pressure graphic 202. For example, view 330 may present the user with monitored patient data 334, as well as pressure and flow waveform graphic 336. In this example embodiment, monitored patient data 334 includes monitored values for the patient's respiratory rate 340, a tidal volume 342, a pressure support value 344, PEEP 346, an I:E ratio 348, a minute volume 350, and an O₂ percentage 352. One or more of the monitored patient data 334 to be displayed in view 330 may be predefined or selected by the user. In addition, one or more of the monitored values may correspond to settings for the particular parameters, as discussed above regarding FIG. 8.

Pressure and flow waveform graphic 336 may illustrate waveforms indicating the monitored airway pressure, gas flow, and/or one or more other parameters over time. In embodiments that illustrate multiple parameters (e.g., both an airway pressure and an gas flow), the two waveforms may be illustrated on the same graphic (as shown in FIG. 10) or on separate graphics. In some embodiments, the user may select between multiple levels of resolution in displaying the waveform graphs on graphic 336, or select the number of sequential breaths to be displayed, e.g., by pressing a resolution button 354. The user may also be able to select or adjust the scale of graphic 336 along one or more axes.

FIG. 11 illustrates an example of an alarm condition displayed in advanced-waveform view 330, according to an embodiment of the disclosure. During an alarm condition, an alarm message window 360 may be displayed in the same location as the digital monitored data (i.e., region 144 discussed above regarding FIG. 4). In some embodiments, while alarm message window 360 is active, the monitored patient data 334 may still be displayed, but resized to a smaller window or area.

As discussed above, menu region 142 may display various menu items that may be selected by the user, e.g., to access the different views discussed above; to access various settings; to set up a new patient for ventilatory care; to view history and/or alarm logs; to setup, edit and/or view multiple preset breath delivery therapies; and/or to adjust the current breath mode, breath type, and/or breath trigger options.

FIG. 12 illustrates an example view 400 having a menu region 142 displaying various menu items that may be selected by a user, according to an embodiment of the disclosure. In this embodiment, menu region 142 may include a view menu button 402, a main menu button 404, a preset menu button 406, and a breath mode menu button 408. Display 52 may be a touch screen display allowing a user to select buttons 402-408 by touching the screen. In some embodiments, selecting a button 402-408 may open up a new window or drop-down menu including various user selectable options associated with the selected button 402-408, as discussed below.

FIG. 13 illustrates an example view in which view menu button 402 has been selected by a user, according to an embodiment of the disclosure. View menu button 402 may allow a user to select a particular view to display, e.g., one of the four views discussed above. When a user selects view menu button 402, a window 420 may open that displays buttons 422 corresponding to each of the views available for display.

In some embodiments, GUI module 22 may manage user access to particular views displayed in window 420, thereby managing user access to particular values. For example, GUI module 22 may restrict user access to particular views using any suitable restriction technique, e.g., using passwords or access keys, or requiring particular buttons or icons to be pressed simultaneously or in sequence.

In some embodiments, one or more views may have restricted access, while one or more other views may have open or unrestricted access. For example, in the embodiment shown in FIG. 13, the Basic and Simple views may be unrestricted, while the Advanced-Gauge and Advanced-Waveform views may be restricted. Thus, when a user selects the Advanced-Gauge or Advanced-Waveform view, GUI module 22 may require the user to bypass the restriction in order to display the selected view. For example, when a user selects the button 422 corresponding to the Advanced-Gauge or Advanced-Waveform views, a pop-up window may be displayed prompting the user to enter a password, access key, or perform any other act to bypass the restriction. In this manner, GUI module 22 may restrict access to the Advanced-Gauge and Advanced-Waveform views, thereby restricting access to particular ventilation parameters that are accessible only in those views.

FIG. 14 illustrates an example view in which main menu button 404 has been selected by a user, according to an embodiment of the disclosure. Main menu button 404 may allow a user to access various settings or other data such that the user may view, set and/or adjust various settings and/or provide other input. When a user selects main menu button 404, a window 430 may open that displays a menu of buttons 432 corresponding to a menu of settings or other data accessible to the user. In this example embodiment, buttons 432 include a main settings button, an alarm settings button, an apnea settings button, a leak test button, a new patient set-up button, a screen brightness button, and a history/alarm logs button. In some embodiments, one or more of such buttons 432 may be selected only when ventilator 24 is not currently ventilating; when ventilator 24 is currently ventilating, such buttons 432 may be grayed out. For example, in this example, the leak test button and the new patient set-up button may be available for selection only when ventilator 24 is not currently ventilating.

In addition, in some embodiments, the menu of settings and/or other data that may be accessed via menu button 404 may depend on the particular view or the access level of the particular view. For example, selecting menu button 404 in a Level 1 access view (e.g., the Simple view) may provide the user access to a first menu of settings and/or other data, selecting the menu icon in a Level 2 access view (e.g., the Main view) may provide the user access to a second menu of settings and/or other data larger than the first menu of settings and/or other data, and selecting the menu icon in a Level 3 access view (e.g., the Advanced-Gauge or Advanced-Waveform view) may provide the user access to a third menu of settings and/or other data larger than the second menu of settings and/or other data. In some embodiments, buttons 432 corresponding to particular settings and/or other data that are not accessible in a particular view may be grayed out or hidden from the menu displayed when the menu button 404 is selected in that view.

For example, in one embodiment, when menu button 404 is selected in the Advanced-Gauge view or the Advanced-Waveform view, window 430 may include all of the menu buttons 432 for selection by the user. When menu button 404 is selected in the Main view, window 430 may include only a subset of the menu buttons 432 for selection by the user; buttons 432 that are not available via the Main view grayed out or not included. When menu button 404 is selected in the Simple view, window 430 may include even a smaller subset of the menu buttons 432 for selection by the user; buttons 432 that are not available via the Main view grayed out or not included.

FIGS. 15-21 illustrate menus and functions associated with preset menu button 406, according to an embodiment of the disclosure. FIG. 15 illustrates an example view in which preset menu button 406 has been selected by a user, according to an embodiment of the disclosure. Preset menu button 406 may allow a user to setup, edit and/or view one or more preset breath delivery therapies. When a user selects preset menu button 406, a window 440 may open that displays buttons 442 corresponding to preset breath delivery therapies accessible to the user, which in this example embodiment include the following preset breath delivery therapies: Preset 1, Preset 2, Preset 3, and None.

Preset menu button 406 may provide the user (e.g., a respiratory therapist) the flexibility to establish, modify, and/or activate multiple breath delivery therapy configurations for one or more patients. If a particular patient has a need for different settings at night or during an active day, these settings can be pre-established by the user (e.g., a respiratory therapist) and may be easy for the caregiver to select. For example, a respiratory therapist may pre-establish one or more breath delivery therapy configurations for a patient, and a caregiver may then select from these pre-established configurations a particular configuration to use.

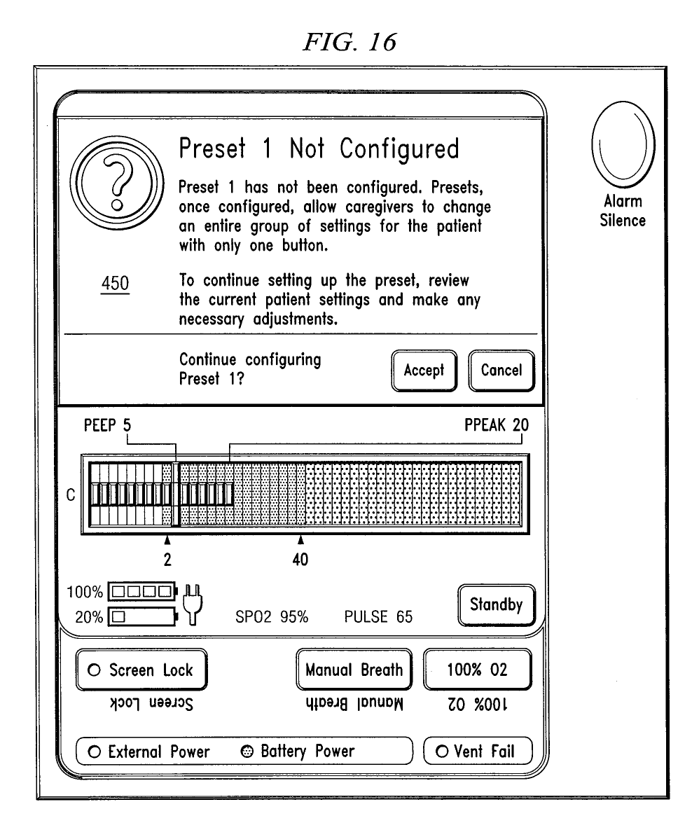

Once ventilator 24 has been initially set-up for a new patient (the initial settings may be referred to as “baseline settings”), the user (e.g., a respiratory therapist) can select preset menu button 406 and then a particular preset, such as “Preset 1 (setup).” When the user selects the particular preset, a window 450 may open that prompts the user regarding how to proceed, as illustrated in FIG. 16. At the “Continue configuring Preset 1?” prompt, if the user selects “Cancel,” window 450 may clear and the display may return to its previous state. Alternatively, if the user selects “Accept,” the user may be guided through one or more preset configuration steps (e.g., using a configuration wizard).

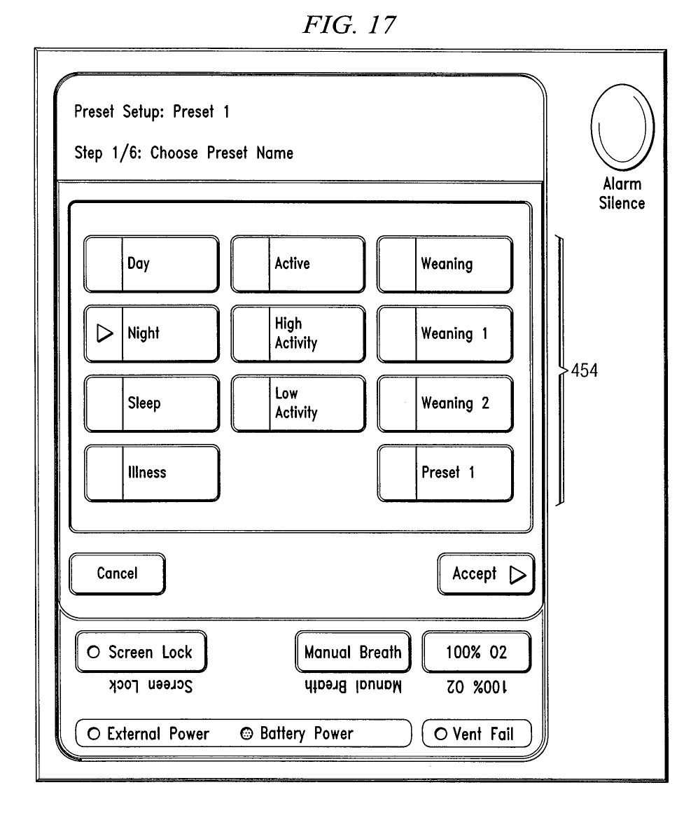

For example, as illustrated in FIG. 17, the user may have the option to label the particular preset button (i.e., the button labeled “Preset 1 (Setup)”) with a name selected from a set of names 454, or using a keypad or other input to type in a desired name. The user may select a name that best corresponds with the breath delivery therapy being configured. Once the user accepts the name selection, the user may be provided access to setup one or more ventilation parameters for that breath delivery therapy. In some embodiments, the user may configure such parameters in any desired order. In other embodiments, GUI module 22 may take the user through a progression or prompts and/or windows in order to configure the breath delivery therapy.

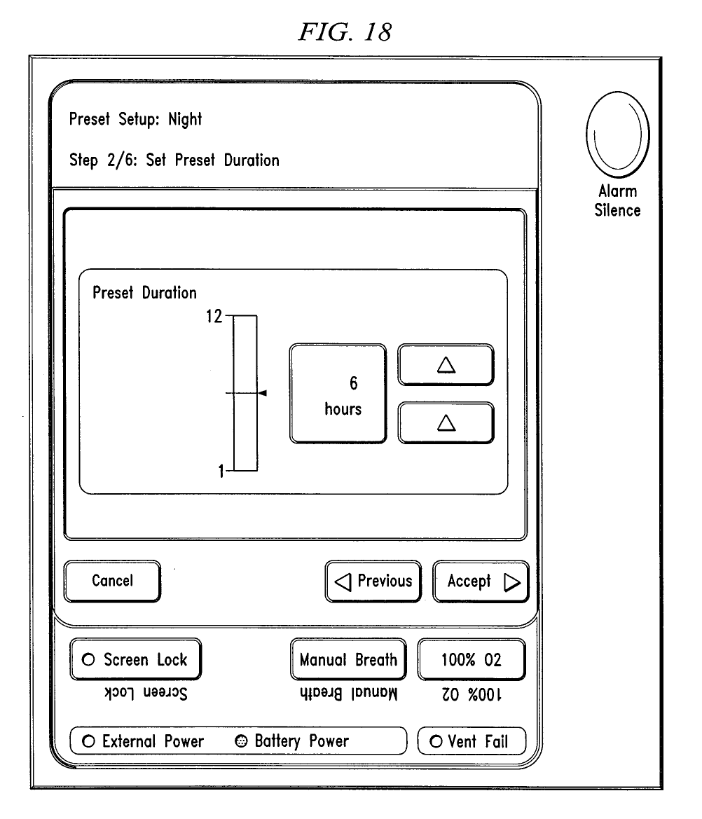

For example, the user may be prompted for a preset duration, e.g., as illustrated in FIG. 18. In operation, once the preset duration time (e.g., 6 hours) has been reached, GUI module 22 may display a visual message and/or an audible tone to remind the caregiver that the preset duration time has been reached. This feature may be provided to help the caregiver remember that the patient is currently being ventilated on preset settings and that they should decide whether to transition the patient to the baseline settings (e.g., by selecting a “preset off” option), a different preset, remain with the currently executing settings, or some other option. In some embodiments, ventilator 24 automatically transitions back to baseline settings when the duration time has been met. In other embodiments, ventilator 24 does not automatically transition back to baseline settings when the duration time has been met.

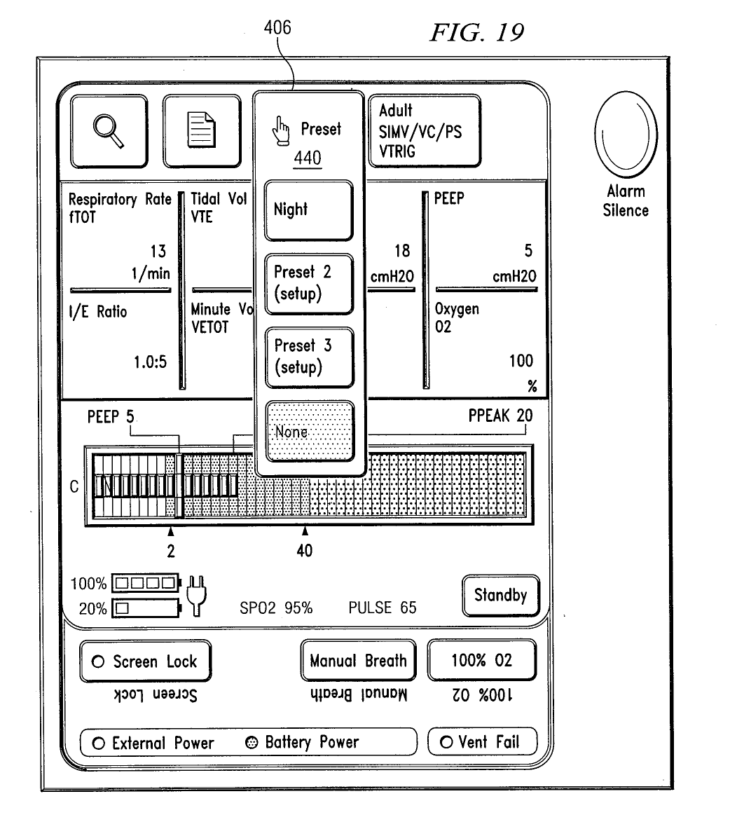

Next, the user may set up the preset settings (e.g., in a similar manner as for a New Patient setup). For example, the user may set values for breath mode settings, main settings, alarm settings, and/or apnea settings, in a selected order or in a predetermined order. The preset values may default to a set of baseline settings so that the user can easily make minor adjustments from the baseline settings. Once the user has completed the preset configuration steps, the preset may become available for selection within the preset window 440 that appears upon selection of preset menu button 406, as indicated in FIG. 19. In this example, the configured preset was named “Night.” In some embodiments, the preset does not automatically initiate after completion of configuration; instead, it must be selected from preset window 440.

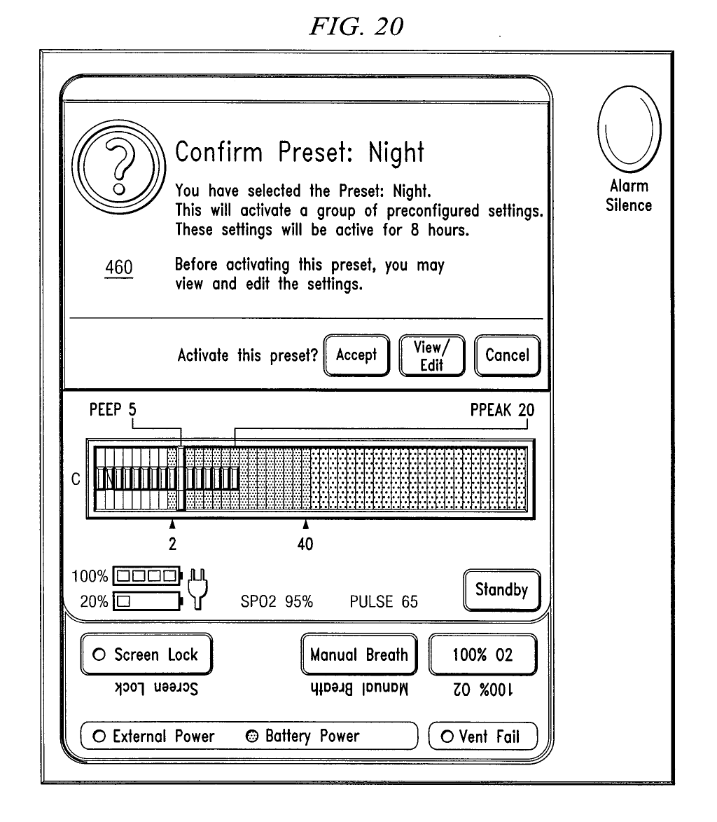

The user (e.g., respiratory therapist) may be able to select, modify, and/or activate a particular preset. FIG. 20 illustrates an example window 460 allowing the user to transition to a particular preset (here, the “Night” preset), view the settings, modify the settings, and/or cancel.

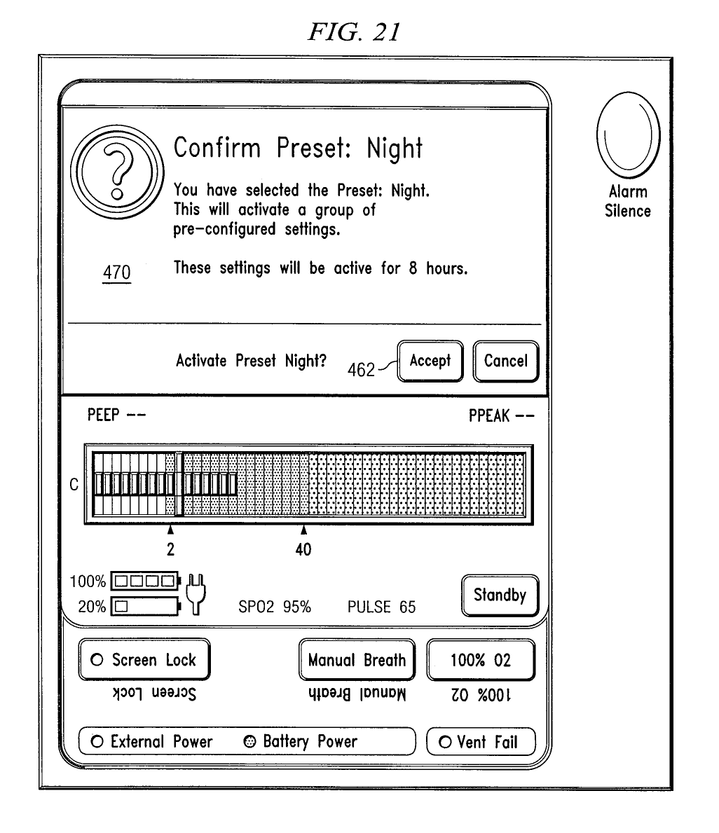

In some embodiments, the caregiver can select and activate a particular preset with three buttons or selections: (1) the preset menu button 406, (2) the particular preset button 442 (e.g., “Night” preset button 442), and (3) a preset confirmation button. In other embodiments, such selection and activation of a preset may require less than three or more than three buttons or selections. FIG. 21 illustrates a confirmation window 470 allowing a caregiver to either transition to a preset or cancel. The “Accept” button 462 may act as the preset confirmation button discussed above.

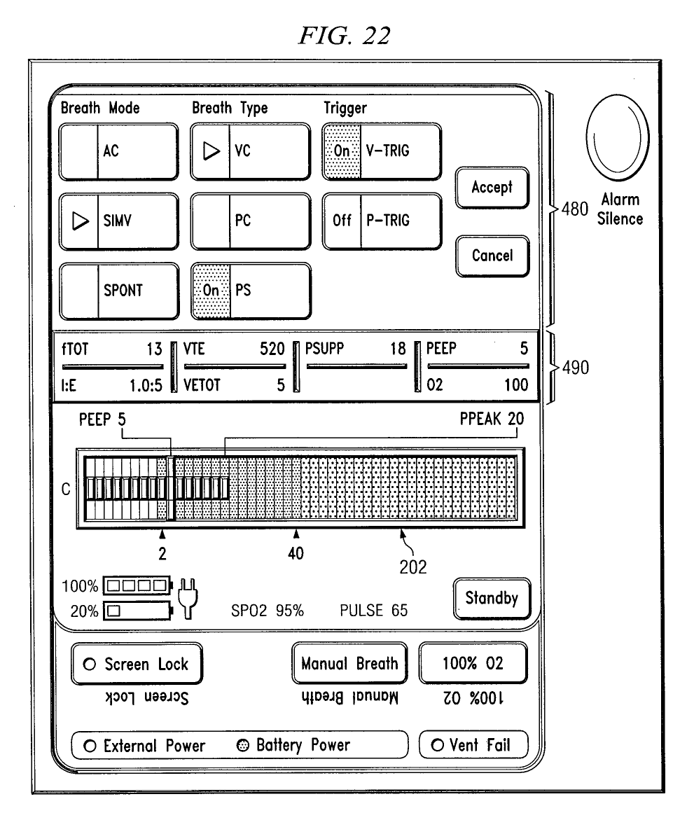

FIG. 22 illustrates an example view in which breath mode menu button 408 has been selected by a user, according to an embodiment of the disclosure. Breath mode menu button 408 may allow a user to adjust the current breath mode, breath type, and/or breath trigger options. When a user selects breath mode menu button 408, a window 480 may open that displays various breath mode options. The breath mode window 480 may appear in the same location as the monitored data. In some embodiments, the monitored data is still displayed, but moved to a smaller window 490 below the breath mode options.

In any of the displays or views discussed herein, some or all dedicated keys and/or on-screen button may include a graphic icon and/or text identifying the purpose of the button to the user. These graphic icons or text may enhance the ease of use for what may otherwise be a confusing array of user inputs. Moreover, the use of graphic icons and/or text to identify the function of dynamically-generated on-screen buttons may provide for virtually unlimited opportunities to add functions to multi-level GUI module 22 by replacing, upgrading, or otherwise modifying GUI software 46, e.g., as new functions are desired by users of the system. Additionally, the use of graphic icons may overcome the potential problem of identifying the functions of a button where language comprehension may be a problem, such as the use of the ventilator in a country where English is not readily understood.

It will be appreciated that while the disclosure is particularly described in the context of a ventilator display, the apparatuses, techniques, and methods disclosed herein may be similarly applied in other contexts, e.g., displays for any other medical devices. Additionally, it should be understood that various changes, substitutions and alterations can be made herein without departing from the spirit and scope of the disclosure as illustrated by the following claims.

What is claimed is:

1. A ventilator system, comprising:
a programmable ventilator configured for ventilating a patient based on settings for a plurality of ventilation parameters; and
a multi-level graphic user interface (GUI) communicatively coupled to the ventilator and configured to display a view menu allowing a user to select from multiple different views providing different levels of user access to the ventilation parameters, the multiple different views including:
a first view displaying values for a first set of the ventilation parameters, each value comprising either a monitored value or a setting for a ventilation parameter, the first view further providing user access for adjusting the setting for at least one of the first set of the ventilation parameters; and
a second view displaying values for a second set of the ventilation parameters, each value comprising either a monitored value or a setting for a ventilation parameter, the second view further providing access for adjusting the setting for at least one of the second set of the ventilation parameters, wherein the second set of ventilation parameters is larger than the first set of ventilation parameters.

2. A ventilator system according to claim 1, wherein the multi-level GUI is at least partially integrated with the ventilator.

3. A ventilator system according to claim 1, wherein the multiple different views further include a third view displaying settings for a third set of the ventilation parameters, wherein the third set of ventilation parameters is smaller than both the first and second sets of ventilation parameters.

4. A ventilator system according to claim 3, wherein the third view does not provide access for adjusting the settings for the third set of the ventilation parameters.

5. A ventilator system according to claim 1, wherein providing access for adjusting a ventilation parameter comprises displaying at least one of:
one or more one-touch icons identifying particular ventilation parameters, wherein each one-touch icon may be selected by a user in order to adjust the setting for the ventilation parameter identified by the selected one-touch icon; and
a menu icon that may be selected by the user in order to display one or more menus including parameter icons identifying a set of ventilation parameters, wherein each parameter icon may be selected the user in order to adjust the setting for ventilation parameter identified by the selected parameter icon.

6. A ventilator system according to claim 5, wherein:
the first view includes one or more one-touch icons identifying at least one of the first set of the ventilation parameters; and
the second view does not include one-touch icons identifying any of the second set of the ventilation parameters.

7. A ventilator system according to claim 5, wherein the second view includes a menu icon that may be selected by the user in order to display one or more menus including parameter icons identifying at least one of the second set of the ventilation parameters.

8. A ventilator system according to claim 1, wherein:
the first view includes a first ventilation graphic;
the second view includes a second ventilation graphic;
each of the first and second ventilation graphics comprises one of:
a waveform graphic indicating at least one of a graphical waveform indicating monitored pressure data and a graphical waveform indicating monitored flow data; and
a gauge graphic including a gauge having an indicator that dynamically advances and retreats to indicate at least one of monitored pressure data and monitored flow data.

9. A ventilator system according to claim 1, wherein at least one of the first and second views includes an airway pressure gauge graphic, the airway pressure gauge graphic comprising a graphical representation of the patient's airway pressure during one or more breath cycles, the graphical representation including a gauge having an indicator that dynamically advances and retreats based on the patient's airway pressure.

10. A ventilator system according to claim 9, wherein the airway pressure gauge graphic indicates a minimum pressure threshold setting, a maximum pressure threshold setting, a positive end expiratory pressure (PEEP) setting, and a peak pressure value.

11. A ventilator system according to claim 1, wherein:
the first and second views are displayed via a touch screen display; and
the ventilator system further comprises one or more continuously available user controls located separately from the touch screen display, the one or more continuously available user controls including:
an alarm silence/reset control for silencing an alarm for a particular period of time;
a screen lock control for deactivating interaction via the touch screen display;
a manual breath control for causing the ventilator to deliver a breath according to current breath settings of the ventilator; and
a 100% O₂ control for causing the ventilator to deliver 100% oxygen to the patient for a particular period of time.

12. A ventilator system according to claim 1, wherein the multi-level GUI includes a touch screen display, the ventilator system further comprising a control device for deactivating user interaction via the touch screen display.

13. A ventilator system according to claim 1, wherein:
the ventilator is configured to receive power from one or more batteries and from an external power source; and
the multi-level GUI is configured to display at least one of:
an indication of the charge status of each of the one or more batteries; and
an indication of whether the ventilator is connected to the external power source.

14. A ventilator system according to claim 1, wherein the view menu allows the user to select from at least three different views generally corresponding to different types of users.

15. A ventilator system according to claim 1, wherein, during an alarm condition, an alarm notification is displayed and at least a portion of the one or more ventilation parameters displayed in the selected view are automatically resized to provide an area for the alarm notification.

16. A ventilator system according to claim 1, wherein the multi-level GUI includes a touch screen display including:
a menu region that displays multiple menus selectable by the user; and
a data region that displays at least one of the following:
settings for one or more ventilation parameters; and
monitored data regarding one or more ventilation parameters;
wherein each of the multiple different views includes a different set of data displayed in the data region.

17. A ventilator system according to claim 16, wherein, during an alarm condition, an alarm notification is displayed in the data region and at least a portion of the data previously displayed in the data region is automatically resized to provide an area for the alarm notification.

18. A ventilator system according to claim 16, wherein the touch screen display further includes a battery/oximeter data region that displays at least one of the following:
status data regarding one or more ventilator batteries; and
oximeter data received from a pulse oximeter.

19. A ventilator system according to claim 1, wherein the multi-level GUI includes a touch screen display including a menu region that displays at least two of the following:
a view menu button for accessing the view menu for allowing the user to select from the multiple different views;
a main menu button for accessing one or more menus of the ventilation parameters, the one or more menus including at least one of a main setting menu, an alarm setting menu, an apnea settings menu, a new patient menu, a leak test menu, and a history menu;
a preset menu button for accessing a preset menu for allowing the user to setup or edit one or more preset breath delivery therapies; and
a breath mode menu button for accessing a breath mode menu for allowing the user to adjust at least one of a current breath mode, a breath type, and one or more breath trigger options.

20. A ventilator system according to claim 1, wherein the multi-level GUI includes a touch screen display including a menu region that displays at least two of the following:
a main settings menu for displaying and adjusting one or more of the ventilation parameters;
an alarm settings menu for displaying and adjusting alarm limits for one or more monitored ventilation parameters;
an apnea settings menu for displaying and adjusting one or more ventilation parameters regarding failure of the patient to initiate a breath within a particular amount of time;
a patient type and breath settings menu for displaying and designating a patient type and one or more breath settings;
a leak test menu for displaying steps for determining whether a patient circuit is securely attached to the ventilator and is free of leaks; and
a history menu for viewing logs of events and alarms during a particular period of time.

21. A ventilator system, comprising:
a ventilator configured for ventilating a patient based on settings for a plurality of ventilation parameters; and
a graphic user interface (GUI) for use with the ventilator, the GUI including a touch screen display configured to display multiple different views;
wherein the multiple different views provide different levels of user access for adjusting one or more of the ventilation parameters;
wherein the multiple different views include at least one restricted access view; and
wherein the GUI is configured to manage user access to each restricted access view by requiring a user to enter particular input in order to access the restricted access view.

22. A ventilator system according to claim 21, wherein:
each of the multiple views includes a selectable menu icon configured to provide user access to one or more options;
selecting the menu icon in a first view provides user access to a first set of options; and
selecting the menu icon in a second view provides user access to a second set of options larger than the first set of options.

23. A ventilator system according to claim 22, wherein:
the first view is not a restricted access view; and
the second view is restricted access view.

24. A ventilator system according to claim 21, wherein the particular input for accessing a restricted access view comprises at least one of a password, an access key, and a combination of buttons pressed simultaneously or in sequence.

25. A multi-level graphic user interface (GUI) for use with a ventilator system, the multi-level GUI including a touch screen configured to display a view menu allowing a user to select from multiple different views providing different levels of user access to a plurality of ventilation parameters, the multiple different views including:
a first view displaying values for a first set of the ventilation parameters, each value comprising either a monitored value or a setting for a ventilation parameter, the first view further providing user access for adjusting the setting for at least one of the first set of the ventilation parameters; and
a second view displaying values for a second set of the ventilation parameters, each value comprising either a monitored value or a setting for a ventilation parameter, the second view further providing access for adjusting the setting for at least one of the second set of the ventilation parameters, wherein the second set of ventilation parameters is larger than the first set of ventilation parameters.

26. Logic encoded in media and, when executed by a processor, operable to display a view menu allowing a user to select from multiple different views providing different levels of user access to a plurality of ventilation parameters, the multiple different views including:
a first view displaying values for a first set of the ventilation parameters, each value comprising either a monitored value or a setting for a ventilation parameter, the first view further providing user access for adjusting the setting for at least one of the first set of the ventilation parameters; and
a second view displaying values for a second set of the ventilation parameters, each value comprising either a monitored value or a setting for a ventilation parameter, the second view further providing access for adjusting the setting for at least one of the second set of the ventilation parameters, wherein the second set of ventilation parameters is larger than the first set of ventilation parameters.

27. A ventilator system, comprising:
ventilation means for ventilating a patient based on settings for a plurality of ventilation parameters; and
display means for displaying a view menu allowing a user to select from multiple different views providing different levels of user access to the ventilation parameters, the multiple different views including:
a first view displaying values for a first set of the ventilation parameters, each value comprising either a monitored value or a setting for a ventilation parameter, the first view further providing user access for adjusting the setting for at least one of the first set of the ventilation parameters; and
a second view displaying values for a second set of the ventilation parameters, each value comprising either a monitored value or a setting for a ventilation parameter, the second view further providing access for adjusting the setting for at least one of the second set of the ventilation parameters, wherein the second set of ventilation parameters is larger than the first set of ventilation parameters.
